(19) United States
(12) Patent Application Publication
(10) Pub. No.: US 2020/0188511 A1
(43) Pub. Date: Jun. 18, 2020

(54) METHODS OF IMPROVING EFFICACY OF ALLERGY VACCINES

(71) Applicants: ANERGIS S.A., Epalinges (CH); MYMETICS SA, Epalinges (CH)

(72) Inventors: Alexander Kettner, Lausanne (CH); Vincent Charlon, Saint Martin D'uriage (FR); Vanya Beltrami, Veyrier (CH); Antonius Johannes Henrikus Stegmann, Rijnsburg (NL); Sylvain Fleury, Bottens (CH); Mario Amacker, Schmitten (CH)

(21) Appl. No.: 16/712,605

(22) Filed: Dec. 12, 2019

Related U.S. Application Data
(60) Provisional application No. 62/778,850, filed on Dec. 12, 2018.

(51) Int. Cl. A61K 39/36; A61K 9/127; A61K 39/39; C12N 7/00; A61K 47/69

(52) U.S. Cl. CPC A61K 39/36; A61K 9/1275; A61K 39/39; A61K 2039/525; A61K 47/6901; A61K 47/6913; C12N 2760/16042; C12N 7/00

(57) ABSTRACT

Provided are specific immunotherapy methods for allergies in which one or more peptides specific for the allergy being treated is administered to the patient incorporated within a virosome and in the presence of a Toll-like receptor (TLR) agonist.

### CROSS-REFERENCE TO RELATED APPLICATION

This application claims the priority benefit of U.S. Patent Application No. 62/778,850 filed Dec. 12, 2018, which is incorporated herein by reference in its entirety.

### BACKGROUND OF THE INVENTION

### Incorporation by Reference of Material Submitted Electronically

Incorporated by reference in its entirety is a computer-readable sequence listing submitted concurrently herewith and identified as follows:

### Incorporation by Reference of Material Submitted Electronically

Incorporated by reference in its entirety is a computer-readable sequence listing submitted concurrently herewith and identified as follows: File name:53777A_Seqlisting.txt; Size: 6,183 bytes; Created: Dec. 12, 2019.

### FIELD OF THE INVENTION

The present invention relates generally to in vivo methods and compositions designed for allergen-specific immunotherapy. The compositions include contiguous overlapping peptides which together comprise some or all of the entire amino acid sequence of an allergen.

### BACKGROUND OF THE INVENTION

IgE⋅mediated allergic disease appears to be very common particularly in industrialized countries where up to one quarter of the population is affected by allergic rhinitis. (Settipane, R. A., Allergy Asthma Proc, 22(4):185-9 (2001)). Furthermore people suffering from allergic rhinitis show a lower quality of life than healthy one, (Bousquet, J., et al., J Allergy Clin Immunol, 94(2):182-8 (1994)) with only a few going into remission spontaneously.

Approximately 25% of all allergic patients respond to tree pollen. Among those, 90% show reactivity with birch pollen extract on cutaneous tests (Skin Prick Tests, SPT). Allergies are triggered by environmental proteins of known peptide sequence and for birch pollen allergy most patients show hypersensitivity to Bet v 1, the major birch pollen allergen. Bet v 1 is part of a protein family playing an important role in plant defense and thus Bet v 1 cross-reacting proteins were found in a number of plants. (Breiteneder, H. et al., J Allergy Clin Immunol, 113(5):821-30 (2004)). In addition, allergy to birch pollen is very often related to allergies to other trees of the Fagales family and with certain food allergies, like those to hazel nut, apple, melon and peach. (Son, D. Y. et al., Eur J Nutr, 38(4):201-15 (1999) and Jahn-Schmid et al., J Allergy Clin Immunol, 116(1):213-9 (2005)).

The only treatment directed to the cause of IgE-mediated allergy is specific immunotherapy (SIT). The treatment consists in injecting increasing doses of allergens for extended periods of time (three to five years) to induce tolerance in the allergic patient. Several studies showed the benefit of this therapy on the allergic response, in particular upon long-term treatment. (Drachenberg, K. J. et al., Allergol Immunopathol, 31(2):77-82 (2003) and Dam Petersen, K. et al., Allergol Immunopathol 33(5)264-269 (2005)). However, a number of side effects were observed particularly during ultra rush therapies, where up to 30% of the patients have to be treated for allergic symptoms during the course of therapy. (Birnbaum et al., Clin. Exp. Allergy, 33(1):58-64 (2003)). There is thus a strong medical need for an alternative to SIT in the form of a shorter treatment with acceptable safety.

Different approaches have been tested to improve the safety and efficacy of SIT. Formulations or existing extracts have been improved by adding adjuvants, like Monophosphory lipid A or MPL (Allergy Therapeutics), (Drachenberg, K. J. et al., Allergol Immunopathol, 31(5):270-7 (2003)) DNA sequences (Hartl, A. et al., Allergy, 59(1):65-73 (2004)) or bacteriophage combined with CpG (Martinez Gomez, J. M. et al., Pharm. Res., 24(10):1927-35 (2007)) which increase the Th1 immune response, thus allowing possible reductions in the amount of allergen extract. Defined allergens were used instead of whole extracts. In the case of birch pollen, a clinical trial with recombinant Bet v 1 has shown efficacy equivalent to whole birch pollen extract (Pauli, G. et al., J. Allergy Clin. Immunol, 122(5):951-60 (2008)).

To diminish the occurrence of allergic symptoms resulting from treatment, different groups explored the use of products with hypoallergenic potential, namely showing reduced IgE binding. In particular, peptides encompassing a restricted number of T-cell epitopes were used for allergen immunotherapy of cat dander with limited efficacy (Campbell, J D et al., J Exp Med., 206(7):1535-47 (2009)). However, allergens harbor a great variety of T cell epitopes partly dependent on the HLA type of the patient. For example, T cell epitopes were found scattered throughout the Bet v 1 sequence, except for a short region (Jahn-Schmid B. et al., J Allergy Clin Immunol, 116(1):213-9 (2005)). Thus an efficient immunotherapy product should preferably contain the complete sequence of the allergen rather than selected T-cell epitopes.

The use of fragments of allergens remains attractive, based on the evidence that human IgE recognize mainly non-contiguous epitopes which may be separated by fragmentation of the allergen. As used herein “fragment” refers both to a peptide derived from digestion of a larger polypeptide or protein and also to a synthesized peptide which is engineered based on the primary amino acid sequence of a given protein. Two contiguous fragments of Bet v 1 or trimeric forms of Bet v 1 were tested in a phase I study in human and showed a trend towards improvement of well being but provided no significant improvement in symptom medication scores (Niederberger, V. et al., Proc Natl Acad Sci USA, 101(2):14677-82 (2004)). In that study, however, a number of adverse events were observed, the majority of which occurred hours after the injections (Purohit, A. et al, Clin Exp Allergy (2008)). Three fragments of the major allergen of bee venom, namely phospholipase A2, were also tested in human, showing an excellent safety due to lowered IgE binding while eliciting elevated levels of IgG4 and IL-10 (Fellrath et al., J. Allergy Clin. Immunol, 111:854-861 (2003)). Of interest to the present invention is the disclosure of U.S. Pat. No. 7,923,209, the disclosure of which is incorporated here, which describes methods of selecting contiguous overlapping peptides (COPs) for treatment of allergy which together form the entire amino acid sequence of an allergen, thus providing all possible T cell epitopes of the allergen, while having lowered IgE binding. Such selected fragments show a reduced ability to reform the original tertiary structure of the allergen, if any, resulting in a reduced ability to bind IgE and therefore to elicit allergic reactions in humans.

U.S. Pat. No. 8,343,503, the disclosure of which is incorporated herein by reference in its entirely, provides COPs from the sequence of the major allergen of birch pollen Bet v 1 allergen for the treatment of allergic patients by specific immunotherapy (SIT). U.S. Pat. Nos. 8,703,144, 9,005,627, 9,193,773 and 9,808,503 the disclosures of which are all incorporated by reference describe respectively, COPs for treatment of patients allergic to dust mite allergen, ragweed pollen, and birch pollen respectively,

Of interest to the present invention are virus-like particles which resemble viruses but lack viral genetic material and are non-infectious. Virus-like particles are useful for the delivery of therapeutic compositions and are also useful as vaccines through their presentation of viral membrane fusion proteins.

Virosomes are lipid-based carrier containing viral integral membrane fusion proteins present in a lipid bilayer membrane and are useful as drug and vaccine delivery vehicles. For example, U.S. Pat. No. 9,216,156 the disclosure of which is incorporated by reference is directed to virosome-like vesicles comprising gp41-derived antigens for inducing an immune response against gp41 protein or a human immunodeficiency virus (HIV). Various adjuvants are proposed for increasing the immunostimulatory effects of the virosome-like vesicles including aluminum salts, aluminum phosphate gels, mycobacteria, peptides, keyhole limpet hemocyanin, muramyl dipeptides and tripeptide derivatives, monophosphoryl lipid A, interleukin-2 (IL-2), IL-12, GM-CSF, ligands from the chemokine family, a lipoprotein of Gram-positive bacteria, a yeast cell wall component, a double-stranded RNA, a lipopolysaccharide of Gram-negative bacteria, flagellin, a U-rich single-stranded viral RNA, a CpG containing DNA, a Suppressor Of Cytokine Signaling small interfering RNA (SOCS siRNA), mellitin-derived peptides, a panDR epitope (PADRE), ligands activating Toll-like receptors (TLRs) and mixtures thereof.

Toll-like receptors (TLRs) are immune receptors that are expressed on the membranes of various immune cells. TLRs bind to foreign entities and recruit proteins important to antigen-specific acquired immunity. There exists an extensive family of TLRs with classes of identified mammalian TLRs numbering from TLR1 to TLR13 recognizing different types of ligands. Also of interest to the present invention are TLR agonists which are known to stimulate the activities of various classes of TLRs. Toll-like receptor agonists are known for use in cancer therapy (see Adams, Immunotherapy 1(6) 949-964 (Nov. 1, 2009)) and U.S. Pat. No. 8,636,979 the disclosure of which is incorporated by reference describes TLR4 and TLR9 receptor agonists for prophylactic treatment of septic complications of post-traumatic systemic immunodepression. See also, Shen et al., Scandinavian J of Immunology 67, 560-568 (2008) which reports that the R-848 (Resiquimod) which is a pure Th1 adjuvant acts as TLR7 and TLR8 agonist and inhibits IgE and IgG1 synthesis by acting directly on B lymphocytes. Further, Chesne et al., Immunol Allergy Clin N Am 36:125-145 (2016) reports the use of a variety of adjuvants for enhancing allergen immunotherapy efficacy including aluminum-based compounds such as alum, TLR agonists, probiotics attenuated mycobacteria and bacterial products, and Vitamin D.

Other lipid vesicles for presentation of antigens include those described in U.S. Pat. No. 9,750,803 the disclosure of which is incorporated by reference, which comprise cross-linked multilamellar vesicles containing an antigen agent in the vesicle core and in between a lipid bilayer. The vesicles are also said to incorporate adjuvants in between the internal lipid bilayers including monophosphoryl lipid A (MPLA) which is a TLR4 agonist and R-848 which is a TLR7 and TLR8 agonist.

Kamphuis et al., PLoS opne Vol. 7, Issue 5, 1-12 (May 2012) “Immunogenicity and Protective Capacity of a Virosomal Respiratory Syncytial Virus Vaccine Adjuvanted with Monophosphoryl Lipid A in Mice” discloses a vaccine for Respiratory Syncytial Virus (RSV) comprising reconstituted RSV viral envelopes (virosomes) incorporated with MPLA adjuvant to enhance immunogenicity and to skew the immune response towards a Th1 phenotype.

Also of interest is Lederhofer, et al. Pharm. Res 35: 172 (2018) “Development of a Virosomal RSV Vaccine Containing 3D-PHAD® Adjuvant: Formulation, Composition, and Long-Term Stability” which discloses a virosomal RSV vaccine with a 3D-PHAD® (synthetic Monophosphoryl 3-Deacyl Lipid A, Avanti Polar Lipids) adjuvant.

Despite the recent advances made in specific immunotherapy and particularly those regarding the use of contiguous overlapping peptides there remains a desire in the art for safer and even more effective methods of immunotherapy with reduced risk of anaphylaxis.

### SUMMARY OF THE INVENTION

The present invention relates to improved methods of specific immunotherapy (SIT) against allergies to an allergen. According to one aspect of the invention a method of SIT is provided comprising the administration of desensitizing allergen peptides (allergen fragments) incorporated within virus-like particles and preferably virosomes in the presence of pattern recognition receptor (PRR) ligands preferably Toll-like receptor (TLR) agonists. It is contemplated that such PRR ligands and TLR agonists can be mixed and administered in solution with the virosomes but it is preferred that the virosomes contain the TLR agonists such as externally wherein they are passively adsorbed on the surface of the virosomes. More preferred are constructions wherein the TLR agonists are included within the lumen of the virosome or lipid bilayer.

According to one aspect of the invention the peptides are integrated into the lipid bilayer of the virus-like particle or virosome. They may be added as lipo-peptides or attached to a lipophilic anchor already present in the virosome structure. According to one preferred aspect of the invention it is contemplated that the peptides be incorporated into the virus-like particle or virosome using click chemistry which allows for specific binding of the peptide to a selected counterpart structure. According to a further aspect of the invention peptides can be lipidated at various amino acids. Particularly preferred lipidation sites for peptides include at their N-terminus where the amino group can be as reactive as the amino groups present on Lysine residues which can also be lipidated as well as at their C-terminus. The peptides can also be lipidated at other amino acid residues. Also contemplated is the use of lipopeptides of the structure, lipid-linker-N-terminus-peptide which can be integrated into the preferred virosomes of the invention. It is also contemplated that the zwitterionic lipid 1-Palmitoyl-2-Oleoyl-sn-Glycero-3-Phosphoethanolamine (POPE) can be used as the lipid attached to the N-terminus of the peptides and COPs. The incorporation of peptides and COPs with other lipophilic and amphiphilic molecules is also contemplated.

The present invention is particularly useful where the one or more peptides comprise a plurality of contiguous overlapping peptides (COPs) comprising some or all of the entire amino acid sequence of the allergen being treated for and is particularly useful where the COPs comprise the entire amino acid sequence of the allergen the allergy to which is being treated for. A further preferred aspect of the invention is that wherein the reactivity of said COPS to IgE antibodies of subjects who are allergic to said allergen is reduced or eliminated, while the reactivity with the T lymphocytes from subjects who are allergic to said allergen is retained. As used herein the statement that reactivity to IgE antibodies is eliminated is understood by those of skill in the immunology art that such reactivity is reduced by three or four or more logs to a level at which it is clinically irrelevant or by which it is undetectable by ordinary measurement techniques. Those of ordinary skill would appreciate that the peptides including peptides making up sets of COPs need not have the exact sequence of the corresponding amino acid sequence of the allergen to which they are directed. Thus, appropriately designed peptides having 95% and even 90% or greater sequence identify to the allergen of interest can be used according to the invention wherein the reactivity of the peptide to IgE antibodies of subjects allergic to the allergen of interest is reduced and/or eliminated while reactivity with the T lymphocytes from subjects who are allergic to said allergen is retained are contemplated for use according to the invention.

Suitable virus like particles for use in practice of the invention include Virus like particles consisting of Qb phage protein (Schmitz et al. J. Exp. Med. 2009), synthetic virus like particles such as described by Boato et al. (Angew. Chem. Int. Ed. 2007), Immune stimulating complexes (ISCOMs) and virosomes including virosomes derived from RSV and according to a preferred aspect of the invention virosomes derived from different strains of influenza such as those available from Mymetics SA.

Pattern recognition receptor (PRR) ligands including Toll-like receptor (TLR) agonists useful in practice of the invention include those specific for TLRs generally but particularly preferred ones include those specific for TLR2, TLR4, TLR7, TLR8 and TLR9 known to be of special interest in allergies. It is also contemplated that TLR agonists can be fused with one another so as to be multifunctional. Those of skill in the art would appreciate that some TLR agonists, such as CL413 discussed below, have agonistic activities directed to more than one class of TLRs. Particularly useful TLR agonists include commercially available ones such as 3D-PHAD® (Monophosphoryl 3-Deacyl Lipid A (Avanti Polar Lipids) a TLR4 agonist); CL413 ((S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl) methyl) aniline Adilipoline™ (Invivogen) as a dual TLR2 and TLR7 agonist.); CL531 ((S-(2,3-bis(palmitoyloxy)-(2RS)propyl)-(R)-cysteinyl-(S)-seryl-(S)-lysyl-Ne-(4-((6-amino-2-(butylamino)-8-hydroxy-9H-purin-9-yl)methyl) benzylamido)(S)-lysyl-(S)-lysyl-(S)-lysine) (Invivogen) as another dual TLR2 and TLR7 agonist); R-848 (Resiquimod) an imidazoquinoline compound, acting on both, TLR7 and TLR8; Single stranded RNA (ssRNA) as an agonist of TLR7 and TLR8 and CpG oligodeoxynucleotides that bind to TLR9.

According to a further aspect of the invention other pattern recognition receptor (PRR) ligands are expected to function in the role of TLR agonists including C-type lectin receptors (CLR): such as trehalose 6,6′-dibehenate (TDB) that binds to Mincle.

Provided herein are not only methods for SIT but also compositions of matter and pharmaceutical compositions comprising one or more peptides specific for an allergy incorporated within a virus-like particle and/or one or more peptides specific for an allergy in the presence of a Toll-like receptor agonist. Also provided are the uses of such compositions of matter in the preparation of medicaments for specific immunotherapy

The methods of the invention are useful in treating a number of different allergies to various allergens. For example, the allergens include, but are not limited to, plant pollens, grass pollens, tree pollens, weed pollens, insect venom, dust mite proteins, animal dander, saliva, fungal spores and food allergens (i.e., peanut, milk, gluten and egg). The patients treated can include all mammals but in particular is selected from the group consisting of humans, dogs, cats, pigs, horses, rats and mice with treatment of humans being most significant and preferred.

Preferred methods and materials are described herein for the treatment of birch pollen allergies including allergies to the Bet v 1 and Bet v 2 birch pollen allergens and preferred aspects of the invention include methods wherein the peptides are selected from the group consisting of Aller T1 (SEQ ID NO 1), Aller T2 (SEQ ID NO 2) and Aller T3 (SEQ ID NO 3). According to one aspect of the invention the at least one of the peptides has the sequence of Aller T2 shifted by the truncation of its N-terminal Asn (N) residue. Nevertheless, it is contemplated that the methods of the invention will be useful in the treatment of any of a wide variety of allergies to other allergens.

The therapeutic compositions of the invention can be administered by any of a variety of means known to those of ordinary skill in the art. In various embodiments, the administration is carried out by parenteral, e.g., skin prick, intravenous, intradermal, subcutaneous, intra muscular, oral, nasal, mucosal (e.g., inhalation), transdermal (topical), transmucosal, lymph node and rectal administration. Those skilled in the art will recognize that any means of administration can be employed.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 depicts SDS-PAGE analysis of COPS conjugated to POPE and COPs virosomal compositions;

FIG. 2 depicts IgE responses to Bet v 1 in mice treated with the COPs compositions;

FIG. 3 depicts IgE responses to Aller T1, Aller T2 and Aller T3 in mice treated with the COPs compositions;

FIG. 4 depicts Bet v 1 specific IgG2a (Th1) responses in mice treated with the COPs compositions;

FIG. 5 depicts Bet v 1 specific IgG1 (Th2) responses in mice treated with the COPs compositions;

FIG. 6 depicts the log ratios of IgG2a/IgG1 (Th1 vs. Th2 responses) for the test compositions;

FIG. 7 depicts the IgG1 (Th2) responses to Aller T1, Aller T2 and Aller T3;

FIG. 8 depicts the IgG2a (Th1) responses to Aller T1, Aller T2 and Aller T3; and

FIG. 9 depicts the body temperature for the test animals upon the last injection at Day 57 for each of the test compositions.

FIG. 10 depicts HPLC profiles of lipidated Bet v 1 COPs

### DETAILED DESCRIPTION OF THE INVENTION

The present invention relates to improved methods of specific immunotherapy (SIT) against allergies to an allergen. According to one aspect of the invention a method of SIT is provided comprising the administration of desensitizing allergen peptides incorporated within virus-like particles and preferably virosomes. According to another aspect of the invention desensitizing allergen peptides are administered to patients in need thereof in the presence of Toll-like receptor (TLR) agonists and according to a further aspect of the invention, allergen peptides are administered to the patient incorporated within a virus-like particle and in the presence of a TLR agonist.

Virosomes are particles composed of a lipid bilayer membrane containing one or more integral membrane proteins of viruses. Virosomes can be the reconstituted membranes of enveloped viruses, artificial vesicles made from bilayer-forming lipids plus one or more viral integral membrane proteins, or vesicles produced by cells.

Virosomes for use in practice of the invention can be produced by solubilizing the membranes of enveloped viruses by means of a detergent or short-chain phospholipid, followed by removal of the short-chain phospholipid or detergent, resulting in reconstitution of the membrane (WO2004/071492, Stegmann T. et al., 1987, EMBO J. 6, 2651-2659). Such virosomes can be expanded by the addition of additional lipids and other hydrophobic materials. Virosomes can also be produced by integrating one or more integral membrane proteins of a virus into an existing lipid bilayer membrane or by mixing one or more integral viral membrane protein of a virus, solubilized in a detergent or short-chain phospholipid with lipids, followed by removal of the detergent. Virosomes can also be produced by expressing one or more integral viral membrane proteins in a suitable host, allowing the formation and secretion of membraneous vesicles containing the integral viral membrane proteins.

Virosomes may be made to contain genetic material such as DNA or RNA. Virosomes may be made to contain proteins or peptides attached to the viral membrane or integrated into the virosomal lumen. These peptides or proteins may be, but are not limited to, antigens from viruses, bacteria, parasites or other any pathogens, allergens, proteins from tumor cells, or peptides derived from proteins expressed by tumor cells. Soluble proteins or peripheral membrane proteins, peptides, nucleic acids and other water-soluble substances can be included in the lumen of the virosome. Peptides or proteins can be adsorbed non-covalently on the surface of virosomes by electrostatic or hydrophobic interactions, or attached covalently to the virosomal membrane through hydrophobic domains, after grafting lipids, fatty acids, and other hydrophobic tails onto the proteins or peptides, and therefore integrated into the lipid bilayer of the virosomes.

The virosomal membrane can be coated with poly(ethylene)glycol or modified by glycosylation. Reporter molecules, for example fluorescent reporter molecules can be incorporated into the virosomal membrane, for example for the measurement of membrane fusion.

Virosomes can be used for the delivery of nucleic acids, proteins, toxins, or other substances into cells. An important feature of virosomes is that they are particles of the size that is efficiently taken up by phagocytic cells of the immune system, and they closely mimic the composition, surface architecture and functional activities, particularly the membrane fusion activity, of the native viral envelope. Virosomes of different sizes may be obtained. The size of the virosome depends on the nature and amount of the viral proteins and the composition of the lipid bilayer. Influenza virosomes typically measure 90-150 nm in diameter. In comparison, virosomes based on the RSV have a mean diameter of about 70 nm, whereas virosomes derived from Herpes virus are between 200 and 500 nm in diameter.

For vaccines, virosomes can be delivered by injection, for example by subcutaneous, intramuscular or intravenous injection. Formulations of virosomes have also been developed for oral, nasal, vaginal or anal delivery.

Virosomes may be stabilized or preserved by spray-drying or freeze-drying. Spray-drying or freeze-drying may be done in the presence of additives such as sugars, either present in the solution containing the virosomes or present within the virosomes, or both.

Most frequently, virosomal vaccines are the reconstituted membranes of influenza virus, to which lipids, adjuvants, and proteins or peptide may have been added, to produce a lipid bilayer membrane that includes said molecules in addition to the influenza membrane fusion protein, hemagglutinin (HA). These virosomes resemble influenza virus on the outside, but do not contain any of the viral genetic material, and can therefore not induce infection. Said virosomes are the ideal shape and size for uptake by antigen-presenting cells, and virosomal vaccination results in humoral and cellular immune responses. Humoral immune responses are typically elicited by antigens carried on the outside of the virosomes.

Influenza virosomes are taken up by antigen-presenting cells through receptor-mediated endocytosis. Fusion between the virosomal and the endosomal membrane is induced by the low pH inside the endosome, resulting in the release of virosomal proteins or peptides into the cytoplasm of the cell, and presentation of virosome-included peptides on MHC-I complexes on the surface of the antigen-presenting cell. Virosomal degradation in endosomes produced MHC-II presented peptides. Thus, virosome vaccines stimulate both arms of the immune system. Virosomal vaccines have also been produced from other enveloped viruses, such as respiratory syncytial virus, to exploit other entry pathways into antigen-presenting cells.

Adjuvants, for steering or enhancing an immunological response, can be included in the membrane of virosomes through hydrophobic domains, by grafting to the membrane, or included in the lumen of the virosomes, or simply mixed with virosomes. Amphiphilic adjuvants can also be incorporated into the membrane of virosomes by providing them from a suitable organic solvent.

Amphiphilic adjuvants in the membrane of virosomes to further improve the capacity of virosomal vaccine formulations to stimulate the immune response following injection or intranasal application of virosomes. See for instance WO2004/110486, wherein virus is solubilized with a detergent or short-chain phospholipid followed by viral nucleocapsid removal. Thereafter, the adjuvant dissolved in the same detergent or short-chain phospholipid, is added to the solubilized viral membranes to incorporate the adjuvant in the virosomes. The detergent or short-chain phospholipid is then removed, resulting in the formation of virosomes that include at least the viral membrane proteins and lipids and the adjuvants. Amphiphilic adjuvants incorporated into the virosomal membrane in this fashion have been shown to be stably integrated in the membrane (Stegmann, T et al. Vaccine 2010; 28(34): 5543-50; WO2004/110486) and enhance or alter the immune response following vaccination with these virosomes in preclinical trials (Kamphuis, T. et al. Plos One 2012; 7 (5):e36812).

The adjuvant can also be added to an already formed virosomal membrane by dissolving the adjuvant in suitable organic solvent that is miscible with water, such as DMSO, and contacting the adjuvant/organic solvent mixture with the virosomes, which results in the adjuvant being present in the outer leaflet of the virosome only, still is available to interact with the receptor present on the cells of the immune system, but halving the potential toxic side effects of the adjuvant (WO 2016/603961).

The antigen/adjuvant ratio profoundly affects the immune response following vaccination. For example, for respiratory syncytial virus (RSV) virosomes containing a monophosphoryl lipid A adjuvant, is was found that a threshold concentration of the adjuvant was capable of skewing the immune response from a dominant Th2 response to a more balanced Th1/Th2 response (Kamphuis, T. et al. Plos One 2012; 7 (5):e36812).

It is to be understood that the terminology used herein is for the purpose of describing particular embodiments only and is not intended to limit the scope of the present invention. As used herein and in the claims, the singular forms “a,” “and” and “the” include plural referents unless the context clearly dictates otherwise.

The terms “human leukocyte antigen” and “HLA” is here defined as a genetic fingerprint on white blood cells and platelets, composed of proteins that play a critical role in activating the body's immune system to respond to foreign organisms.

The term “plurality of contiguous overlapping peptide fragments (OPF)” is here defined as at least one, but most likely two, three, four, or five, contiguous overlapping peptide fragments. For example, the schematic below shows an example of a plurality of contiguous overlapping peptide fragments, if the alphabet was a 26 residue peptide, and the plurality contained four overlapping peptides: OPF₁₋₆, OPF₄₋₁₅, OPF₁₃₋₂₂ and OPF₂₀₋₂₆:
ABCDEF=OPF₁₋₆
DEFGHIJKLMNO=OPF₄₋₁₅
MNOPQRSTUV=OPF₁₃₋₂₂
TUVWXYZ=OPF₂₀₋₂₆

The term “hypersensitive” is here defined as abnormally susceptible physiologically to a specific agent via IgE-mediated mechanisms (as an antigen or drug). Such antigen is in the present specification and claims called an allergen.

The term “hyposensitive” is here defined as not being sensitive to a specific agent (as an antigen or drug). Such antigen is in the present specification and claims called an allergen.

The terms “desensitize”, “immunological tolerance” or “tolerance” are here defined as to make (a sensitized or hypersensitive individual) insensitive or nonreactive to a sensitizing agent (as an antigen or drug) by a reduction in immunological reactivity of a host towards specific tolerated antigen(s). Such antigen is in the present specification and claims called an allergen.

The term “positive-control” is here defined as a native allergen that when applied to the skin will produce a positive reaction i.e. a red area, the flare and a raised spot, the wheal, at the test site if IgE antibody is present. Apart native allergens, examples of positive-controls include pharmacological agents such as, but not limited to, histamine. The optimal positive-control is the allergen itself in its native confirmation.

The term “negative-control” is here defined as a composition that when applied to the skin, should not produce, at 15 minutes, a response with a flare >5 mm when the injected volume of solution (50 μl) produces spontaneously a papule of 5 mm. Negative-controls include OPF diluent, albumin solution or saline (salt-water) solution.

The term “papule” is here defined as a small circumscribed, superficial, solid elevation of the skin. When related to allergens, it is usually measured by a wheal and flare reaction which is an outward spreading zone of reddening flare followed rapidly by a wheal (swelling) at the site of introduction of the allergen.

The term “erythema” is here defined as redness of the skin produced by congestion of the capillaries, which may result from a variety of causes.

The term “isolated” or “purified” peptide fragments or biologically active portion thereof is substantially free of material (e.g., other, contaminating proteins) from the cell suspension, tissue source, or serum preparation from which the allergen peptide fragments are derived, or substantially free from chemical precursors or other chemicals when chemically synthesized. The language “substantially free of other material” includes preparations of the allergen-derived peptide fragments in which the peptide fragments are separated from cellular components of the cells from which it is isolated or recombinantly produced. In one embodiment, the peptide fragments having less than about 30% (by dry weight) of non-allergen protein (also referred to herein as a “contaminating protein”), more preferably less than about 20% of non-allergen protein, still more preferably less than about 10% of non-allergen protein, and most preferably less than about 5% non-allergen protein. When the allergen-derived peptide fragments are recombinantly produced, it is also preferably substantially free of culture medium, i.e., culture medium represents less than about 20%, more preferably less than about 10%, and most preferably less than about 5% of the volume of the overlapping peptides preparation.

The language “substantially free of chemical precursors or other chemicals” includes preparations of the allergen-derived peptide fragments in which the peptide fragments are separated from chemical precursors or other chemicals which are involved in the synthesis of the protein. In one embodiment, the language “substantially free of chemical precursors or other chemicals” includes preparations of the allergen-derived peptide fragments having less than about 30% (by dry weight) of chemical precursors or non-allergen chemicals, more preferably less than about 20% chemical precursors or non-allergen chemicals, still more preferably less than about 10% chemical precursors or non-allergen chemicals, and most preferably less than about 5% chemical precursors or non-allergen chemicals.

Manipulations of the sequences included within the scope of the invention may be made at the peptide level. Included within the scope of the present invention are peptide fragments (derivative or analog thereof) that are modified during or after translation or synthesis (e.g., by glycosylation, acetylation, phosphorylation, amidation, derivatization by known protecting/blocking groups, proteolytic cleavage, linkage to an antibody molecule or other cellular ligand, and the like). Any of the numerous chemical modification methods known within the art may be utilized including, but not limited to, specific chemical cleavage by cyanogen bromide, trypsin, chymotrypsin, papain, V8 protease, NaBH4, acetylation, formylation, oxidation, reduction, metabolic synthesis in the presence of tunicamycin, etc. In a specific embodiment, sequences of a peptide are modified to include a fluorescent label Allergen-derived peptide fragments, analogs, derivatives, and variants thereof can be chemically synthesized. For example, a peptide fragment corresponding to a portion of an allergen protein that includes a desired domain or that mediates a desired activity in vitro, may be synthesized by use of a peptide synthesizer. The amino acid sequence of a protein isolated from the natural source, may be determined, e.g., by direct sequencing of the isolated protein. The protein may also be analyzed by hydrophilicity analysis (see, Hopp and Woods, PNAS USA 78:3824, 1981) which can be used to identify the hydrophobic and hydrophilic regions of the protein, thus aiding in the design of peptides for experimental manipulation, such as in binding experiments, antibody synthesis, etc. Secondary structural analysis may also be performed to identify regions of a peptide that adopt specific structural motifs. (See, Chou and Fasman, Biochem, 13:222, 1974). Manipulation, translation, secondary structure prediction, hydrophilicity and hydrophobicity profiles, open reading frame prediction and plotting, and determination of sequence homologies, can be accomplished using computer software programs available in the art. Other methods of structural analysis including, but not limited to, X-ray crystallography (see, Engstrom Biochem Exp Biol 11:7, 1974); mass spectroscopy and gas chromatography (see, Methods in Protein Science J. Wiley and Sons, New York, N.Y. 1997); computer modeling (see, Fletterick and Zoller, eds., 1986, Computer Graphics and Molecular Modeling, In: Current Communications in Molecular Biology, Cold Spring Harbor Laboratory Press, Cold Spring Harbor, N.Y.); optical rotary dispersion (ORD) and circular dichroism (CD) may also be used. For example, measurement of circular dichroism may be used to determine the linearity of candidate peptides for use as COPs.

The peptide fragments, derivatives and other variants described herein, can be modified. Thus, the invention includes, e.g., myristylated, glycosylated, palmitoylated and phosphorylated peptides and their derivatives.

Conservative amino acid substitutions can be made in the peptide fragments at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, in a peptide fragment with a conservative amino acid substitution a predicted non-essential amino acid residue in the allergen-derived fragment is preferably replaced with another amino acid residue from the same side chain family. Alternatively, in another embodiment, mutations can be introduced randomly along all or part of the allergen coding sequence, to identify mutants that retain T cell stimulating activity but have lower or reduced/weak levels of IgE stimulating activity.

In some embodiments, a mutant allergen peptide fragment can be assayed for (1) the ability to stimulate or induce T cell proliferation or (2) the ability, or lack of, to bind IgE antibodies from, e.g., the sera of an individual hypersensitive to the allergen. The terms “stimulate” or “induce” are used interchangeably herein.

A peptide fragment or combination of overlapping peptide fragments derived from a protein allergen, can be tested to determine whether the peptide will produce local or systemic symptoms that are related to a Type I reaction. This reaction involves the interaction of antigen with antibody of the immunoglobulin class IgE, which attaches to the host cells in the skin and other tissues (mast cells, basophils, platelets, and eosinophils). An antigen encounter results in release of the cell contents, including active molecules such as histamine, heparin, serotonin, and other vasoactive substances, producing local or systemic symptoms that are manifest within minutes to a few hours following antigen-IgE interaction. IgE binding activity of candidate COPs can also be measured by means of ELISA assays using IgEs specific for selected polypeptide allergens having less than a selected maximum binding affinity. According to one aspect of the invention, ELISA assays may be conducted on candidate COPS wherein COPs are selected which have a binding activity for IgE's reactive with the selected polypeptide allergen which is less than three times the standard deviation of a negative control in a conventional ELISA assay.

T cell stimulating activity can be tested by culturing T cells obtained from an individual sensitive to the allergen proteins and variants described herein (i.e., an individual who has an immune response to the protein allergen or protein antigen) with an allergen protein or variant and determining the presence or absence of proliferation by the T cells in response to the peptide as measured by, for example, incorporation of tritiated thymidine. Stimulation indices for responses by T cells to peptides useful in methods of the invention can be calculated as the maximum counts per minute (cpm) incorporated in response to the peptide divided by the cpm of the control medium. For example, a peptide derived from a protein allergen may have a stimulation index of about 2.0. A stimulation index of at least 2.0 is generally considered positive for purposes of defining peptides useful as immunotherapeutic agents. Preferred peptides or fragments or combinations of overlapping fragments have a stimulation index of at least 2.5, more preferably at least 3.5 and most preferably at least 5.0.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., percent homology equals the number of identical positions divided by the total number of positions times 100).

It is further contemplated that the methods of the invention can be applied to specific allergen chimeric or fusion proteins. As used herein, a specific allergen “chimeric protein” or “fusion protein” comprises, an allergen polypeptide operatively linked to a non-allergen polypeptide. An “allergen polypeptide” refers to a polypeptide having an amino acid sequence corresponding to a specific allergen, whereas a “non-allergen polypeptide” refers to a polypeptide having an amino acid sequence corresponding to a protein which is not substantially homologous to the specific allergen, e.g., a protein which is different from the allergen and which is derived from the same or a different organism. Within a specific allergen fusion protein the allergen polypeptide can correspond to all or a portion of a specific allergen protein. In a preferred embodiment, a specific allergen fusion protein comprises at least one biologically active portion of the specific allergen. The non-allergen polypeptide can be fused to the N-terminus or C-terminus of the allergen polypeptide.

COP fragments useful for the invention can be incorporated into compositions suitable for administration. Such compositions typically include the contiguous overlapping peptide fragments and a pharmaceutically acceptable carrier. The term “pharmaceutically acceptable carrier” refers to a carrier that does not cause an allergic reaction or other untoward effect in subjects to whom it is administered. Suitable pharmaceutically acceptable carriers include, for example, water, saline, phosphate buffered saline, dextrose, glycerol, ethanol, or the like, and combinations thereof. In addition, if desired, the composition can contain minor amounts of auxiliary substances such as wetting or emulsifying agents, and/or pH buffering agents which enhance the effectiveness of the vaccine. Attention is directed to Remington's Pharmaceutical Science by E. W. Martin.

The use of such media and agents for pharmaceutically active substances is well known in the art. Except insofar as any conventional media or agent is incompatible with the active compound, use thereof in the compositions is contemplated. Supplementary active compounds can also be incorporated into the compositions. As used herein, the phrases ‘composition’ and ‘therapeutic composition’ are interchangeable.

Compositions containing contiguous overlapping allergen peptide fragments, or variants thereof can be administered to a patient (such as a human) sensitive to the specific allergen in a form which results in a decrease in the T cell response of the mammal upon subsequent exposure to the protein allergen. As used herein, a decrease or modification of the T cell response of a mammal sensitive to a protein allergen is defined as non-responsiveness or diminution in symptoms to the protein allergen in the patient, as determined by standard clinical procedures (see, Varney et al., British Medical Journal, 302: 265, 1990), including diminution in allergen induced asthmatic conditions. As referred to herein, a diminution in symptoms to an allergen includes any reduction in the allergic response of a patient, such as a human, to the allergen following a treatment regimen with a composition as described herein. This diminution in symptoms may be determined subjectively in a human (e.g., the patient feels more comfortable upon exposure to the allergen), or clinically, such as with a standard skin test or provocation assay.

In addition, administration of the above-described contiguous overlapping allergen peptide fragments or their variants may result in lower levels of IgE stimulation activity. Preferably, administration results in weak IgE stimulating activity. More preferably, administration results in zero IgE stimulating activity. As used herein, weak IgE stimulating activity refers to IgE production and/or cross-linking that is less than the amount of IgE production and/or IL-4 production stimulated by the whole protein allergen.

Administration of the compositions of the present invention to desensitize or tolerize an individual to a protein allergen or other protein antigen can be carried out using procedures, at dosages and for periods of time effective to reduce sensitivity (i.e., to reduce the allergic response) of the individual to a protein allergen or other protein antigen. Effective amounts of the compositions will vary according to factors such as the degree of sensitivity of the individual to the protein allergen, the age, sex, and weight of the individual, and the ability of the peptide(s) to elicit a tollerogenic response in the individual. Dosage regimens may be adjusted to provide the optimum therapeutic response. For example, several divided doses may be administered daily or the dose may be proportionally reduced as indicated by the exigencies of the therapeutic situation.

A composition of the invention is formulated to be compatible with its intended route of administration. Examples of routes of administration include parenteral, e.g., skin prick, intravenous, intradermal, subcutaneous, intramuscular, oral, nasal, mucosal (e.g., inhalation), transdermal (topical), transmucosal, lymph node and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of toxicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Administration, e.g., subcutaneous administration, of an allergen-derived overlapping peptide or variant peptides as described herein to a patient, such as a human, can tolerize or anergize appropriate T cell subpopulations such that they become unresponsive to the protein allergen and do not participate in stimulating an immune response upon subsequent exposure. In addition, administration of such a peptide may modify the lymphokine secretion profile as compared with exposure to the naturally-occurring protein allergen or portion thereof (e.g., result in a decrease of IL-4 and/or an increase in IL-10, TGFβ, and IFN-γ). Furthermore, exposure to the peptide may influence T cell subpopulations which normally participate in the response to the allergen such that these T cells, when re-exposed to the native allergen, are secreting high levels of IL-10, TGFβ, or IFN-γ, instead of high levels of IL-4 or IL-5. This immune deviation of T cell subpopulations may ameliorate or reduce the ability of an individual's immune system to stimulate the usual immune response at the site of normal exposure to the allergen, resulting in a diminution in allergic symptoms.

Compositions suitable for injectable use include sterile aqueous solutions (where the peptides or protein are water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., overlapping peptide fragments) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The compositions can be included in a container, pack, or dispenser together with instructions for administration.

It is also possible to modify the structure of peptides useful in methods of the invention for such purposes as increasing solubility, enhancing therapeutic or preventive efficacy, or stability (e.g., shelf life ex vivo, and resistance to proteolytic degradation in vivo). A modified peptide can be produced in which the amino acid sequence has been altered, such as by amino acid substitution, deletion, or addition, to modify immunogenicity and/or reduce allergenicity, or to which a component has been added for the same purpose. For example, the amino acid residues essential to T cell epitope function can be determined using known techniques (e.g., substitution of each residue and determination of presence or absence of T cell reactivity). Those residues shown to be essential can be modified (e.g., replaced by another amino acid whose presence is shown to enhance T cell reactivity), as can those which are not required for T cell reactivity (e.g., by being replaced by another amino acid whose incorporation enhances T cell reactivity but does not diminish binding to relevant MHC molecules). Another example of a modification of peptides is substitution of cysteine residues preferably with alanine, or alternatively with serine or threonine to minimize dimerization via disulfide linkages.

In order to enhance stability and/or reactivity, peptides can also be modified to incorporate one or more polymorphisms in the amino acid sequence of a protein allergen resulting from natural allelic variation. Additionally, D-amino acids, non-natural amino acids or non-amino acid analogues can be substituted or added to produce a modified synthetic peptide within the scope of this invention.

### EXAMPLES

According to these examples, the potential immunizing effect of different Bet v1 COP-virosome formulations were compared to those of a prior-art Bet v1 COP formulation (Aller T) comprising a set of three overlapping peptides with an aluminum hydroxide adjuvant. The compositions were evaluated after five subcutaneous administrations to female CD-1 mice. Specifically, Bet v 1 virosome compositions were prepared comprising three overlapping COP peptides having the sequences of SEQ ID NO: 1 (AllerT1 having 49 amino acids), SEQ ID NO: 2 (AllerT2 having 71 amino acids) and SEQ ID NO: 3 (AllerT3 having 55 amino acids) corresponding to the Bet v 1 birch pollen allergen sequence as published under Swissprot P15494 (SEQ ID NO: 4).

Composition VirA comprised the Bet v 1 virosome compositions above further containing the 3D-PHAD® TLR4 agonist (Monophosphoryl 3-Deacyl Lipid A (Avanti Polar Lipids)

Composition VirB comprised the Bet v 1 virosome compositions above further containing the CL413 TLR2/TLR7 agonist ((S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl) methyl) aniline also named Adilipoline™ (Invivogen), a dual TLR 2 and TLR 7 agonist.)

### Example 1

### Preparation of Virosomes without Added Antigen (Placebo Virosomes)

For a final volume of 2 ml, 10 mg 1,2-dioleyl-sn-glycero-3-phosphatidylcholine (DOPC) were dissolved in 1 ml of 50 mM HEPES pH 7.4, 145 mM NaCl buffer (HN) containing 100 mM octaethyleneglycol-mono-(n-dodecyl)ether (OEG-HN). Inactivated influenza virus (A/Brisbane/59/2007 (H1N1), from Segirus, Australia), containing 0.5 mg hemagglutinin (HA) was centrifuged at 100,000×g for 1 h at 4° C. and the pellet was dissolved in 1 ml of OEG-HN. The detergent solubilized virus was centrifuged at 100,000×g for 1 hr at 18° C., and the supernatant was mixed with the detergent solubilized phospholipid. Virosomes were then formed by detergent removal by shaking the combined solution for one hour with 1 g of wet SM2 Bio-Beads (BioRad, Glattbrugg, Switzerland) at room temperature (RT). The solution was then separated from the beads and added to 1 g of fresh wet SM2 Bio-beads, and again shaken at RT for 1 hr. The resulting virosomes were separated from the beads and filtered with a sterile filter (Millex Durapore PVDF filter unit, 0.22 um, 33 mm) into sterile glass vials and stored at 4° C.

### Example 2

### Preparation of Virosomes with Integrated Bet v1 COP Conjugate Antigen as Intermediate Vaccine

To produce virosomes without added adjuvant, for a final volume of 2 ml, 10 mg DOPC and 2.5-4.0 mg of the heterologous antigen-PE conjugate (Bet v1 COP conjugate as described in example 4) were dissolved in 1 ml of OEG-HN. Inactivated influenza virus (A/Brisbane/59/2007 (H1N1), from Segirus, Australia), containing 0.5 mg hemagglutinin (HA) was centrifuged at 100,000×g for 1 h at 4° C. and the pellet was dissolved in 1 ml of OEG-HN. The detergent solubilized virus was centrifuged at 100,000×g for 1 hr at 18° C., and the supernatant was mixed with the detergent solubilized phospholipid to a final volume of 2 mL. Virosomes were then prepared as described in example 1.

### Example 3

### Preparation of Virosomes with Integrated Bet v1 COP Conjugate Antigen and Added Adjuvant as Intermediate Vaccine

To produce virosomes with Bet v1 COP conjugate and 3D-PHAD®, for a final volume of 2 ml, 10 mg DOPC and 2.5-4.0 mg of the heterologous antigen-PE conjugate (Bet v1 COP conjugate as described in example 4) were dissolved in 1 ml of OEG-HN, and 0.2 ml of 3D-PHAD® (1.0 mg/mL in 100% DMSO) was added. Inactivated influenza virus (A/Brisbane/59/2007 (H1N1), from Seqirus, Australia), containing 0.5 mg hemagglutinin (HA) was centrifuged at 100,000×g for 1 h at 4° C. and the pellet was dissolved in 0.8 ml of OEG-HN. The detergent solubilized virus was centrifuged at 100,000×g for 1 hr at 18° C., and the supernatant was mixed with the detergent solubilized phospholipid to a final volume of 2 mL. Virosomes were then produced as described in example 1.

To produce virosomes with Bet v1 COP conjugate and CL413, for a final volume of 2 ml, 10 mg DOPC and 2.5-4.0 mg of the heterologous antigen-PE conjugate (Bet v1 COP conjugate as described in example 4) were dissolved in 1 ml of OEG-HN, and 0.24 ml of CL413 (1.0 mg/mL in water) was added. Inactivated influenza virus (A/Brisbane/59/2007 (H1N1), from Seqirus, Australia), containing 0.5 mg hemagglutinin (HA) was centrifuged at 100,000×g for 1 h at 4° C. and the pellet was dissolved in 0.8 ml of OEG-HN. The detergent solubilized virus was centrifuged at 100,000×g for 1 hr at 18° C., and the supernatant was mixed with the detergent solubilized phospholipid to a final volume of 2 mL. Virosomes were then produced as described in example 1. The antigen content was analyzed by RP-HPLC.

### Example 4

### Preparation of Antigen-PE Conjugates

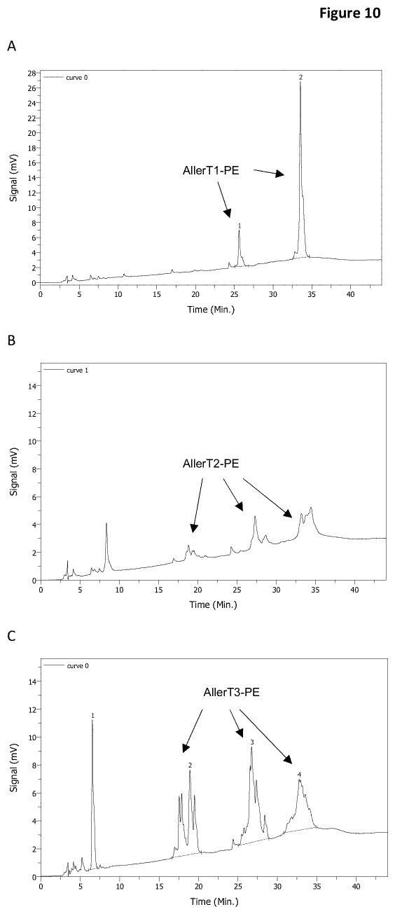

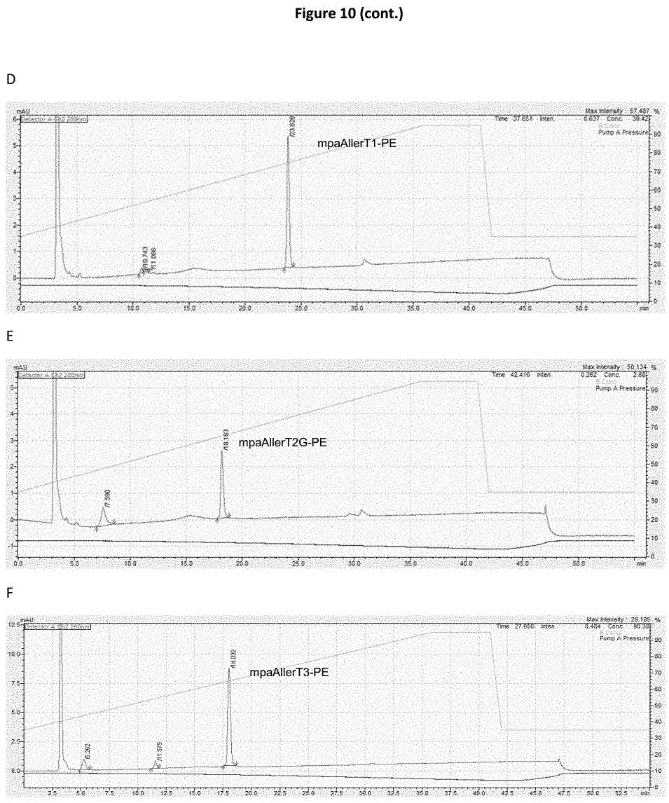

Each antigen (Bet v1 Aller T1, Bet v1 Aller T2, Bet v1 Aller T3) was dissolved separately in 20 mM HCl to obtain a solution of 5 mg/mL. The pH of the solution was afterwards adjusted to 6.5-7.0 by the addition of 1 M NaOH. A solution of 10 mg/mL NHS-POPE was prepared in OEG-HN. The specific antigen solution and the solubilized NHS-POPE solution were mixed at a ratio of 1:2 to 1:10, and incubated at 4° C. or ambient temperature for either 30-90 min with continuous stirring. This conjugation method allows addition of a phospholipid group to N-terminus and lysine residues of the peptides, thus resulting in peptides that may carry multiple phospholipid groups. The antigen conjugation reaction was analyzed and quantified by RP-HPLC (Reverse Phase-High Performance Liquid Chromatography) (FIG. 10 A-C).

Alternatively, Bet v 1 COPs were produced synthetically carrying a maleimide group at the N-terminus. The maleimid group is attached through a linker to the N-terminus of the peptide. Maleimide has a high reactivity to thiol groups. Exploiting this selective reactivity, this modification allows for production of peptides that carry a single phospholipid group at the N-terminus after reacting the maleimide group with a thiol group containing phospholipid such as DPPT (1,2-dipalmitoyl-sn-glycero-3-phosphothioethanol). Each of the maleimide-modified antigens (Bet v1 mpaAllerT1, Bet v1 mpaAllerT2G, Bet v1 mpaAllerT3) was dissolved separately in pure DMSO to obtain a solution of 20 mg/ml. A solution of 10 mg/ml DPPT (1,2-dipalmitoyl-sn-glycero-3-phosphothioethanol) was prepared in pure DMSO. Each specific antigen solution and the DPPT lipid solution were mixed separately at a ratio of 1:3 to 1:10, and incubated at ambient temperature for either 1-12 h with continuous stirring. The antigen conjugation reaction was analyzed and quantified by RP-HPLC (FIG. 10 D-F). Such antigen-PE solutions were used to prepare intermediate virosome solutions for further use.

### Example 5

### Preparation of Final Vaccine with Bet v1 COP Antigens

To produce the final vaccine formulations with the combined Bet v1 antigens, individual virosome preparation with Bet v1 COP antigens with or without adjuvant were combined under aseptic conditions to a final total Bet v1 concentration of 50 μg animal dose (e.g. per 0.1 mL). If necessary, the solution was adjusted with HN buffer to the final required volume. The final vaccine was stored in sterile glass vials at 4° C.

The compositions were compared to a prior art Bet v1 COP formulation (Aller T) comprising a set of three overlapping peptides with an aluminum hydroxide adjuvant wherein the overlapping peptides comprised SEQ ID NO: 1 (AllerT1 having 49 amino acids), SEQ ID NO: 2 (AllerT2 having 71 amino acids) and SEQ ID NO: 3 (AllerT3 having 55 amino acids).

The compositions were further tested against an equimolar mixture of Aller T1, Aller T2 and Aller T3 in the absence of any adjuvant labeled as “no adj”.

For purposes of testing, animals were distributed into seven experimental groups as follows: two groups were treated with VirA (one group at 10 μg/animal and the other at 40 μg/animal) and two additional groups received the same doses of VirB. In the case of the two reference item treated groups, only one dose (40 μg/animal) (high dose) was used. Each experimental group consisted of 20 animals except for those treated with 10 μg/animal (low dose)—this group had 10 females per group. There was an additional group of five females, which were used as baseline control and did not receive any treatment.

The animals from all treated groups were administered subcutaneously on five different days (1, 8, 15, 29 and 57) over the study period. During the whole study, local reactions, mortality, clinical signs, body weight, and food consumption were monitored at defined intervals. In addition, the body temperature was recorded in all animals on the administration days before injection and 30 min after injection in order to determine the potential hypersensitivity reaction induced by the different items. Blood collection was carried out at different days during the study period. Serum was isolated and stored at −80° C.±10 until shipment to the sponsor for the determination of the immunizing effect of the different items.

On the administration days, the injection site of the treated animals were checked under blind conditions prior to and approx. 15, 30, 60, 120 and 180 minutes after treatment.

The reactions of animals will be observed and graded according to the following scales (modified from Sade et al., 2007):

TABLE 1

| Reaction/Sign | Grade |
|---|---|
| No symptoms | 0 |
| Scratching and rubbing around the nose and head | 1 |
| Puffiness around the eyes and mouth, diarrhea | 2 |
| (if any observed), piloerection (strong), reduced activity | |
| and/or decreased activity with increased respiratory rate | |
| Wheezing, labored respiration, cyanosis around the | 3 |
| mouth, tongue or the tail | |
| No activity after prodding or tremor and convulsion | 4 |
| Death | 5 |

Detailed clinical observations in response to treatment were performed at least once weekly until sacrifice. The frequency of the observations were extended when deemed necessary taken into account the onset, duration and severity of toxic signs. Following administration, observations were performed 30 minutes, 1, 3 and 6 hours after injection. Animals from the non-treated group were only observed at the 6 h time point. Any visible clinical signs, discomfort and mortality were recorded in accordance with the humane endpoints guidance document of the OECD. Observations included changes in the skin, eyes and mucous membranes. Alterations in respiratory pattern, behaviour, posture, response to handling and the presence of abnormal movements were also recorded

Rectal Temperature Measurement:

On the administration days, rectal temperature measurements was recorded twice before administration, and approx. 30 min after treatment in all animals, except for in those from the non-treated group, in which only the basal record was taken.

Blood Collection for Immunology Testing:

Blood (approx. 0.3 mL/animal) was collected on days 22, 85 and 106 by retroorbital sinus puncture into tubes without anticoagulant and thereafter blood samples were incubated for approximately 30 min at room temperature. Afterwards, tubes were centrifuged for approx. 10 min at about 1000×g and 5±3° C. in order to isolate serum. Immediately afterwards, each serum sample was stored in an upright position frozen (−80±10° C.) until shipment.

Analysis of antigen specific mouse IgG1, IgG2a and IgE by ELISA.

For the detection of antigen specific IgE, ELISA plates (Maxisorp, Thermo Fisher Inc., Wohlen, Switzerland for Bet v 1 and Immobilizer, Thermofisher, Switzerland for COPs) were coated with of Bet v 1 (2 μg/ml) or COPs (AllerT1, 0.5 μg/ml; AllerT2, 0.25 μg/ml); AllerT3, 0.5 μg/ml) in carbonate buffer. Sera were diluted 1:20 and IgE antibodies were then detected after sequential incubation with biotinylated anti-mouse IgE (clone RME-1, Lucerna, Switzerland) followed by streptavidin-HRP (BD-Biosciences, San Diego, Calif.) and the substrate TMB. For IgG1 and IgG2a, ELISA plates were prepared as for IgE detection.

For the detection of Bet v 1 specific IgG1 and IgG2a, sera were serially diluted 1:5 starting with a first dilution of 1:2′000. IgG1 and IgG2a binding to Bet v 1 was detected after sequential incubation with biotinylated anti-mouse IgG1 (clone LO-MG1-2, Lucerna, Switzerland) or anti-mouse IgG2a (clone RMG2a-62, Lucerna, Switzerland) followed by streptavidin-HRP (BD-Biosciences, San Diego, Calif.) and the substrate TMB. Titer was determined using the highest dilution at which a positive signal was detected. COP specific IgG1 and IgG2a were detected as described above with sera diluted 1:2′000 for detection of AllerT1 specific IgG1 and IgG2a and 1:20′000 for AllerT2 and AllerT3 specific IgG1 and IgG2a.

In vitro assays (IgE ELISA competition and basophil degranulation assays) were conducted on each of the Aller T1, Aller T2 and Aller T3 peptides which have consistently shown absence of detectable IgE binding. These experiments are necessary to ensure that the presentation of the peptides in the Bet v1 COP-Virosomes is not associated with the appearance of unexpected binding to pre-existing Bet v 1-specific IgE.

Competition ELISA.

Bet v 1 at 2 μg/ml was coated overnight on 96-well Nunc Maxisorp® immunoplates (Thermo Fisher Scientific Inc., Wohlen, Switzerland). Sera from birch allergic persons were diluted either ten-fold or twenty-fold (depending on Bet v 1 specific IgE content) and added to serial dilutions of either Bet v 1 or COPs virosomes and incubated for 15 minutes on ice. Serial dilutions of Bet v 1 and COPs virosomes ranged from 10⁻⁶ M to 10⁻¹² M, final concentration. Serum antigen mixtures were then added to the ELISA plates. Biotin Mouse anti-human mAb IgE at 5 μg/ml (BioLegend, San Diego, Calif.) were then added and antibodies were revealed with Streptavidin HRP (BD-Biosciences, San Diego, Calif.) and the substrate TMB.

RP_HPLC:

Lipidated Bet v1 COPs were analyzed by RP-HPLC. The column used was a Interchrom Uptisphere PH HPLC column (250 mm L×4.6 mm ID, 5 μm particle size, 122 Å pore diameter). Eluents A (0.1% Trifluoroacetic acid (TFA) in water) and B (Acetonitrile:Isopropanol 80/20, 0.08% TFA) were used with a flow rate of 1 mL/min. The following gradient was applied: 0-36 min 35-95% B, 36-41 min 95% B, 41-42 min 95-35% B, 42-47 min 35% B. Column oven temperature was set to 50° C. Detection was performed at 280 nm.

Dynamic Light Scattering:

Virosome samples were diluted 1:100 in HN pH 7.4 buffer (50 mM HEPES, 145 mM NaCl). 1 mL was transferred to a PMMA (polymethyl methacrylate) cuvette.

Measurement were performed in a Malvern Zetasizer Nano S instrument. Three measurements were acquired (12×10 seconds each) and averaged. Size of the virosomes (nm) and polydispersity index of the COPs compositions are reported in Table 2 showing dynamic light scattering analysis of virosome compositions.

TABLE 2

| Sample | Size (nm) | Polydispersity index |
|---|---|---|
| T1 virosome 3D-PHAD® | 112.3 | 0.215 |
| T1 virosome CL413 | 117.7 | 0.113 |
| T2 virosome 3D-PHAD® | 83.0 | 0.168 |
| T2 virosome CL413 | 85.4 | 0.138 |
| T3 virosome 3D-PHAD® | 138.6 | 0.271 |
| T3 virosome CL413 | 162.6 | 0.227 |

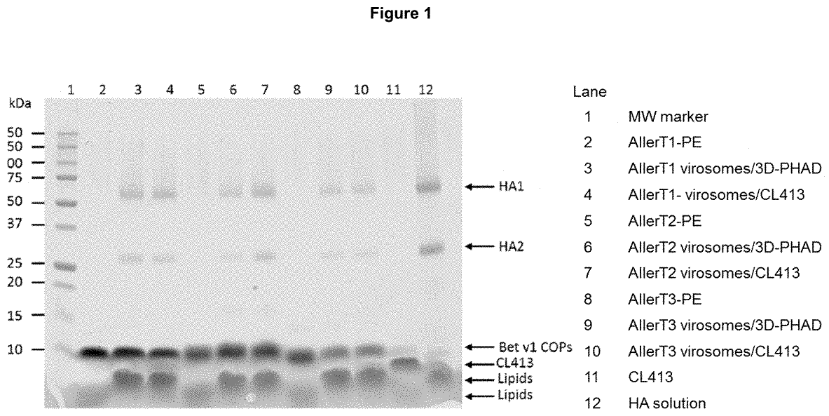

The results are presented in FIGS. 1-10 which show that administration of AllerT led to the development of Bet v 1 specific IgEs (p<0.001) associated with a more pronounced Th2 than Th1 response. In contrast, in the groups receiving Bet v 1 COP-virosomes, no development of Bet v 1 specific IgEs were observed (p<0.001 vs AllerT). With the same dose of Bet v1 COPs there was a strong boost of immunogenicity with a Th1 antibody response which was a hundred times greater than with aluminium hydroxide (p<0.001). The Bet v1 COP-virosomes were well tolerated.

More specifically, FIG. 1 depicts SDS-Page analysis of COPs conjugated to POPE and COPs virosomal preparations. Gel was stained with Coomassie Blue (Instant Blue). Description of samples (˜2.5 μg) applied to Lanes 1-12 is indicated at the right of the graphic. Positions of hemagglutinin1 (HA1), hemaglutinin2 (HA2), lipid conjugated COPs, CL413 and lipids are indicated on the right of the gel. Molecular weights (kDa) are indicated on the left of the gel. Virosomal preparations contain similar amounts of hemagglutinin 1/2 and lipid conjugated COPs as well as free lipid coming from the viral envelope indicating that the lipid conjugated COPs were integrated into the virosomal preparations.

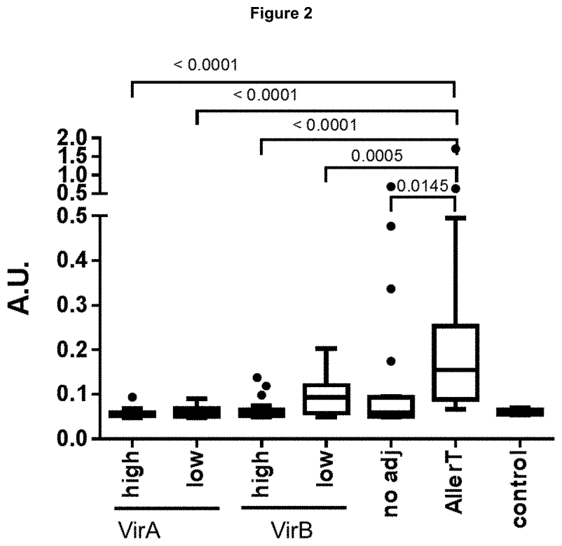

FIG. 2 depicts Bet v 1 specific IgE responses in mice treated with the COPs compositions. IgE responses are expressed as arbitrary units (A.U.) and presented as box and whiskers plots in the style of Tukey with median and interquartiles. P values were determined using the 2-tailed Mann-Whitney test. VirA preparations (high and low dose) did not induce IgE antibodies that recognize Bet v1. IgE response to Bet v 1 in the VirB high dose group is present in low levels in 2 out of 20 animals. Administration of low dose VirB preparation, COPs without adjuvant (no adj) and COPs adsorbed to alhydrogel induced an IgE response against Bet v 1. Strongest IgE response to Bet v 1 was detected in animals treated with COPs adsorbed to alhydrogel. The presence of IgE antibodies that recognize Bet v 1 indicate a sensitization of the animals to Bet v 1 similar to allergy induction in man. Presence of IgE antibodies against Bet v 1 in mice treated with no adjuvanted COPs indicates that COPs have the capacity to sensitize animals and potentially also humans. The sensitization effect is strongly enhanced with alhydrogel as adjuvant. Actually, an increase in Bet v1 specific IgE after treatment with AllerT (COPs adjuvanted with alhydrogel) (Spertini et al. JACI, 2016). Combining COPs with virosomes and TLR4 adjuvant overcome the capacity of COPs to sensitize the animals.

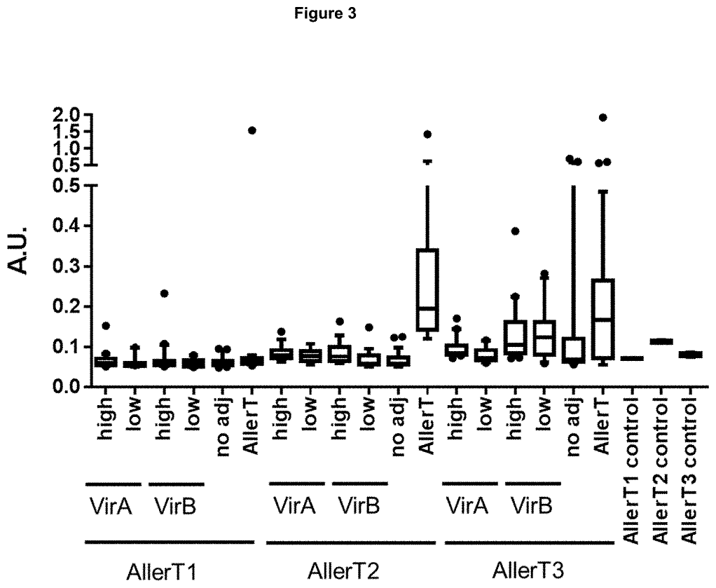

FIG. 3 depicts peptide specific IgE responses in mice treated with the COPs compositions. IgE responses to individual COPs (Aller T1, Aller T2 or Aller T3) are expressed as arbitrary units (A.U.) and presented as box and whiskers plots in the style of Tukey with median and interquartiles. P values were determined using the 2-tailed Mann-Whitney test. Treatment of mice with AllerT induced IgE antibodies that recognized COPs AllerT2 and AllerT3. Furthermore, AllerT3 specific antibodies were induce after treatment with high and low dose VirB (virosomes containing CL413 as adjuvant) as well as COPs without adjuvant. Combining COPs with virosomes and TLR4 adjuvant overcome the capacity of AllerT3 to sensitize the animals. In contrast, VirB preparations, both high and low dose induced AllerT3 specific IgE, indicating that the TLR2/7 agonist did not help to suppress sensitization to AllerT3. Nevertheless, sensitization was lower when compared to AllerT and no sensitization to AllerT2 was observed.

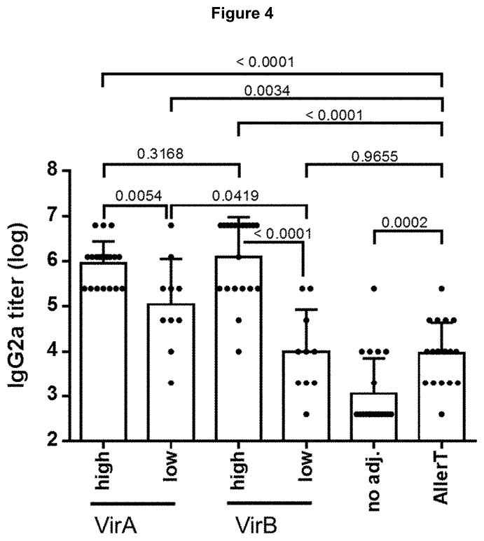

FIG. 4 depicts Bet v 1 specific IgG2a (Th1) responses in mice treated with the COPs compositions. IgG2a responses are presented as log₁₀ transformed titer as mean±standard deviation (SD). P values were determined using the 2-tailed Mann-Whitney test. Both COPs virosome compositions (VirA and VirB) at high and low dose induced a strong IgG2a response to Bet v 1. A 2-log difference was observed when compared to AllerT. These results indicate that the capacity of VirA and VirB to induce Bet v 1 specific IgG2a is strongly enhanced as when COPs were adjuvanted with alhydrogel.

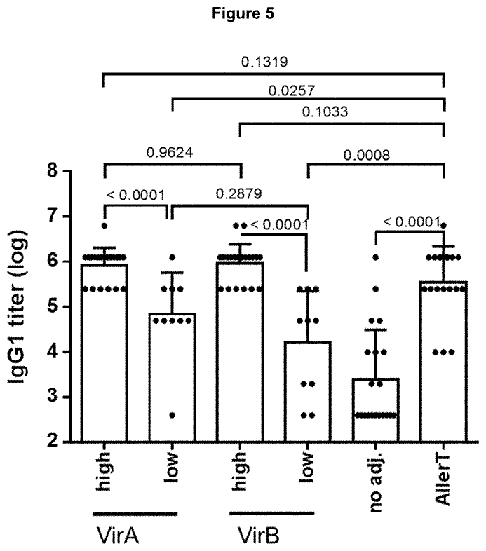

FIG. 5 depicts Bet v 1 specific IgG1 (Th2) responses in mice treated with the COPs compositions. IgG1 responses are presented as log₁₀ transformed titer as mean±standard deviation (SD). P values were determined using the 2-tailed Mann-Whitney test. VirA and VirB compostitions induced the same amount of Bet v1 specific antibodies as AllerT. These results indicate that VirA and VirB compostitions induce the same Th2 type response as AllerT.

Induction of both, IgG2a and IgG1 antibodies that recognize the native allergen, Bet v 1 indicate that the integration of COPs in influenza virosomes with an additional TLR ligand might induce allergen blocking antibodies once administered in the course of AIT in birch pollen allergic patients.

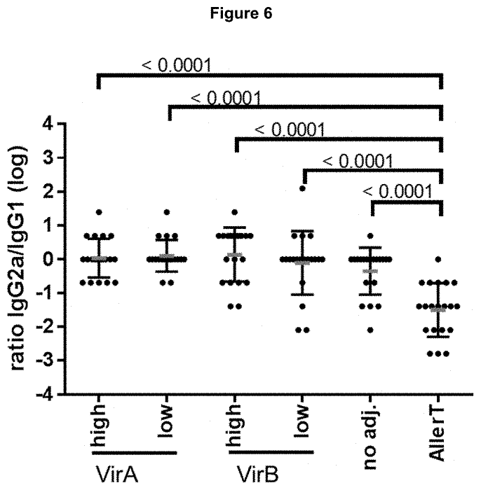

FIG. 6 depicts the log ratios of IgG2a/IgG1 (Th1 vs. Th2 responses) for the test compositions with both high and low doses of the VirA and VirB compositions compared to compositions without adjuvant of adsorbed to alhydrogel. P values were determined using the 2-tailed Mann-Whitney test. These results indicate the Th1 skewed response when COPs were integrated in influenza derived virosomes containing a TLR adjuvant. This type of response is the preferred profile for a product for AIT.

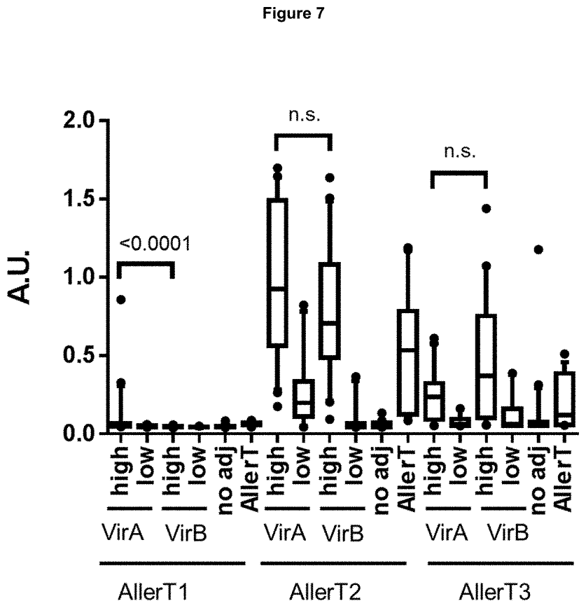

FIG. 7 depicts the IgG1 (Th2) responses to individual COPs (Aller T1, Aller T2 or Aller T3). Results are expressed as arbitrary units (A.U.) and presented as box and whiskers plots in the style of Tukey with median and interquartiles. P values were determined using the 2-tailed Mann-Whitney test. There is no difference of VirA or VirB in the amount of IgG1 specific for AllerT2 or AllerT3. Only VirA induced IgG1 to AllerT1 in few mice. Both VirA and VirB induce a stronger IgG1 response to COPs than AllerT.

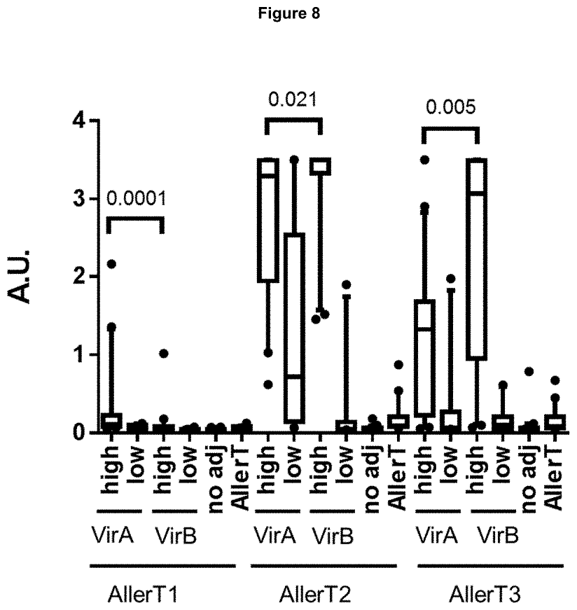

FIG. 8 depicts the IgG2a (Th1) responses to individual COPs (Aller T1, Aller T2 or Aller T3). Results are expressed as arbitrary units (A.U.) and presented as box and whiskers plots in the style of Tukey with median and interquartiles. P values were determined using the 2-tailed Mann-Whitney test. The IgG2a response to individual COPs is rather heterogeneous in the sense that VirA induced more IgG2a to AllerT1, the same amount to AllerT2 and less to AllerT3 when compared to VirB. Both VirA and VirB induced a much stronger IgG2a response than AllerT. These results indicate that VirA and VirB elicit a much stronger Th1 response than AllerT. Of interest is the distinct difference between the two TLR adjuvants to a selective induction of IgG2a responses to individual COPs. This finding indicates together with the results from the IgE response that the two TLR agonists modulate the immune response to the same COPs differently in the sense that there is not only a quantitative but also qualitative difference. Possibly through induction of a different cytokine profile produced by the antigen presenting cells.

The IgG1 and IgG2a response to AllerT1 after treatment with VirA indicates that AllerT1 is immunogenic and might also elicit an immune response in man and thus should be part of the product candidate.

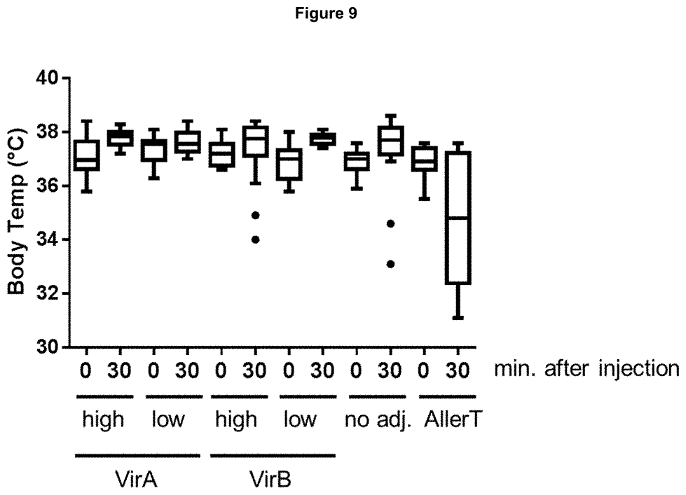

FIG. 9 depicts the body temperature for the test animals upon the last injection at Day 57 for each of the test compositions. Body temperature was measured before (0 min) and 30 minutes after injection of the COPs compositions. Results are presented as box and whiskers plots in the style of Tukey with median and interquartiles. P values were determined using the 2-tailed Mann-Whitney test.

From these experiments it can be concluded that a preferred product is composed of influenza derived virosomes (VirA) with integrated lipidated COPs of SEQ ID: 1, SEQ ID NO: 2 and SEQ ID NO: 3 and 3D-PHAD® as adjuvant. The preferred product candidate did not sensitize mice, reflected by the absence of IgE antibody induction, and represents an improved product for AIT compared to AllerT. The preferred product induced a strong Th1 skewed response, a profile that has been described as desirable for an AIT product. The Th1 response is also indicative of a cellular response (T cells) that is also desirable for an AIT product. The preferred product induced a strong antibody response that recognize the native allergen Bet v 1. This strong antibody response might indicate a possible induction of blocking antibodies in the course of AIT in birch pollen allergic patients.

FIG. 10 depicts the HPLC profiles of lipid conjugated COPs. A, lipidated AllerT1 with lipids attached to N-terminus and to lysine (K) residues. B, lipidated AllerT2 with lipids attached to N-terminus and to lysine (K) residues. C, lipidated AllerT3 with lipids attached to N-terminus and to lysine (K) residues. D, lipidated mpaAllerT1 with lipid attached at N-terminus via 3-maleimidopropionic acid linker. E, lipidated mpaAllerT2G with lipid attached at N-terminus via 3-maleimidopropionic acid linker. F, lipidated mpa AllerT3 with lipid attached at N-terminus via 3-maleimidopropionic acid linker.

These results indicate that lipidation of AllerT1, AllerT2 and AllerT3 leads to multiple conjugates. N-terminal lipidation via maleimide conjugation leads to a single conjugate for mpaAllerT1-PE, mpaAllerT2G-PE and mpaAllerT3-PE respectively.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

SEQUENCE LISTINGS
AllerT1: aa 2-50 of SEQ ID 4
SEQ ID NO: 1
GVFNYETETT SVIPAARLFK AFILDGDNLF PKVAPQAISS
VENIEGNGG
Theoretical pI/Mw: 4.36/5198.82
AllerT2: aa 48-118 of SEQ ID 4
SEQ ID NO: 2
NGGP GTIKKISFPE GFPFKYVKDR VDEVDHTNFK YNYSVIEGGP
IGDTLEKISN EIKIVATPDG GSILKIS
Theoretical pI/Mw: 5.72/7742.76
AllerT3: aa 106-160 of SEQ ID 4
SEQ ID NO: 3
VATPDG GSILKISNKY HTKGDHEVKA EQVKASKEMG
ETLLRAVESY LLAHSDAYN
Theoretical pl/Mw: 6.29/6001.72
Bet v 1 sequence as published under Swissprot
P15494
SEQ ID NO: 4
MGVFNYETET TSVIPAARLF KAFILDGDNL FPKVAPQAIS
SVENIEGNGG PGTIKKISFP EGFPFKYVKD RVDEVDHTNF
KYNYSVIEGG PIGDTLEKIS NEIKIVATPD GGSILKISNK
YHTKGDHEVK AEQVKASKEM GETLLRAVES YLLAHSDAYN
AllerT2G: aa 49-118 of SEQ ID 4
SEQ ID NO: 5
GGP GTIKKISFPE GFPFKYVKDR VDEVDHTNFK YNYSVIEGGP
IGDTLEKISN EIKIVATPDG GSILKIS
Theoretical pI/Mw: 5.72/7628.66
mpaAllerT1: aa 2-50 of SEQ ID 4
SEQ ID NO: 6
mpa-GVFNYETETT SVIPAARLFK AFILDGDNLF PKVAPQAISS
VENIEGNGG
Theoretical pI/Mw: 4.36/5349.9
mpaAllerT2G: aa 49-118 of SEQ ID 4
SEQ ID NO: 7
mpa-GGP GTIKKISFPE GFPFKYVKDR VDEVDHTNFK
YNYSVIEGGP IGDTLEKISN EIKIVATPDG GSILKIS
Theoretical pI/Mw: 5.72/7797.7
mpa AllerT3: aa 106-160 of SEQ ID 4
SEQ ID NO: 8
mpa-VATPDG GSILKISNKY HTKGDHEVKA EQVKASKEMG
ETLLRAVESY LLAHSDAYN
Theoretical pl/Mw: 6.29/6152.8
mpa: 3-maleimidopropionic acid

What is claimed:

1. A method of specific immunotherapy against allergies to an allergen comprising administering to a patient in need thereof one or more peptides specific for the allergy being treated wherein the peptide is administered to the patient incorporated within a virosome and in the presence of a Toll-like receptor (TLR) agonist.

2. The method of claim 1 wherein the one or more peptides comprise a plurality of contiguous overlapping peptides (COPs) comprising some or all of the entire amino acid sequence of the allergen being treated for.

3. The method of claim 2 wherein the COPs comprise the entire amino acid sequence of the allergen being treated for.

4. The method of claim 2 wherein the reactivity of said COPS to IgE antibodies of subjects who are allergic to said allergen is eliminated while the reactivity with the T lymphocytes from subjects who are allergic to said allergen is retained.

5. The method of claim 1 wherein the allergen is birch pollen.

6. The method of claim 5 wherein the allergen is Bet v 1 or Bet v 2.

7. The method of claim 1 wherein the virosome is an influenza virosome.

8. The method of claim 1 wherein the TLR agonist is selected from the group consisting of TLR2, TLR4, TLR7, TLR8 and TLR9 agonists.

9. The method of claim 1 wherein the TLR agonist is Monophosphoryl 3-Deacyl Lipid A (3D-PHAD®) (TLR4) or ((S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl) methyl) aniline (CL413) (TLR2 and TLR7).

10. The method of claim 1 wherein at least one of said peptides is selected from the group consisting of Aller T1 (SEQ ID NO 1), Aller T2 (SEQ ID NO 2) and Aller T3 (SEQ ID NO 3).

11. The method of claim 1 wherein at least one of said peptides has the sequence of Aller T2 shifted by the truncation of its N-terminal Asn (N) residue.

12. The method of claim 1 wherein at least one of said peptides is lipidated.

13. The method of claim 12 wherein at least one of said peptides is lipidated at its N-terminus, its C-terminus and/or at a lysine residue.

14. A composition for specific immunotherapy comprising one or more peptides specific for an allergy incorporated within a virosome in the presence of a Toll-like receptor (TLR) agonist.

15. The composition of claim 14 wherein the one or more peptides comprise a plurality of contiguous overlapping peptides (COPs) comprising some or all of the entire amino acid sequence of the allergen being treated for.

16. The composition of claim 14 wherein the COPs comprise the entire amino acid sequence of the allergen being treated for.

17. The composition of claim 14 wherein the reactivity of said COPS to IgE antibodies of subjects who are allergic to said allergen is eliminated while the reactivity with the T lymphocytes from subjects who are allergic to said allergen is retained.

18. The composition of claim 14 wherein the allergen is birch pollen.

19. The composition of claim 18 wherein the allergen is Bet v 1 or Bet v 2.

20. The composition of claim 14 wherein the virosome is an influenza virosome.

21. The composition of claim 14 wherein the TLR agonist is selected from the group consisting of TLR2, TLR4, TLR7, TLR8 and TLR9 agonists.

22. The composition of claim 21 wherein the TLR agonist is Monophosphoryl 3-Deacyl Lipid A (3D-PHAD®) or ((S-(2,3-bis(palmitoyloxy)-(2RS)propyl)-(R)-cysteinyl-(S)-seryl-(S)-lysyl-(S)-lysyl-(S)-lysyl-(S)-lysyl 4-((6-amino-2-(butylamino)-8-hydroxy-9H-purin-9-yl) methyl) aniline (CL413).

23. The composition of claim 14 wherein at least one of said peptides is selected from the group consisting of Aller T1 (SEQ ID NO 1), Aller T2 (SEQ ID NO 2) and Aller T3 (SEQ ID NO 3).

24. The composition of claim 14 wherein at least one of said peptides has the sequence of Aller T2 shifted by the truncation of its N-terminal Asn (N) residue.

25. The composition of claim 14 wherein at least one of said peptides is lipidated.

26. The composition of claim 14 wherein at least one of said peptides is lipidated at its N-terminus, its C-terminus and/or at a lysine residue.

27. The use of the composition of claim 1 for the manufacture of a medicament for specific immunotherapy for an allergy.


SEQUENCE LISTING (8 sequences)

SEQ ID NO: 1 - protein, 49 aa, Artificial Sequence
  Synthetic Polypeptide
GVFNYETETTSVIPAARLFKAFILDGDNLFPKVAPQAISSVENIEGNGG

SEQ ID NO: 2 - protein, 71 aa, Artificial Sequence
  Synthetic Polypeptide
NGGPGTIKKISFPEGFPFKYVKDRVDEVDHTNFKYNYSVIEGGPIGDTLEKISNEIKIVATPDGGSILKIS

SEQ ID NO: 3 - protein, 55 aa, Artificial Sequence
  Synthetic Polypeptide
VATPDGGSILKISNKYHTKGDHEVKAEQVKASKEMGETLLRAVESYLLAHSDAYN

SEQ ID NO: 4 - protein, 160 aa, Birch Pollen Allergen
MGVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 5 - protein, 70 aa, Birch Pollen Allergen
GGPGTIKKISFPEGFPFKYVKDRVDEVDHTNFKYNYSVIEGGPIGDTLEKISNEIKIVATPDGGSILKIS

SEQ ID NO: 6 - protein, 49 aa, Birch Pollen Allergen; identical to SEQ ID NO: 1
  MISC_FEATURE (1)..(1): 3-maleimidopropionic acid

SEQ ID NO: 7 - protein, 70 aa, Birch Pollen Allergen; identical to SEQ ID NO: 5
  MISC_FEATURE (1)..(1): 3-maleimidopropionic acid

SEQ ID NO: 8 - protein, 55 aa, Birch Pollen Allergen; identical to SEQ ID NO: 3
  MISC_FEATURE (1)..(1): 3-maleimidopropionic acid